(19) United States
(12) Patent Application Publication
(10) Pub. No.: US 2016/0147941 A1
(43) Pub. Date: May 26, 2016

(54) MEDICAL SUPPORT SYSTEM

(71) Applicant: FUJIFILM Corporation, Tokyo (JP)

(72) Inventors: Satoshi UEDA, Tokyo (JP); Yasunori OHTA, Ashigarakami-gun (JP); Ryosuke USAMI, Tokyo (JP)

(73) Assignee: FUJIFILM Corporation, Tokyo (JP)

(21) Appl. No.: 15/010,694

(22) Filed: Jan. 29, 2016

Related U.S. Application Data
(63) Continuation of application No. PCT/JP2014/070032, filed on Jul. 30, 2014.

(30) Foreign Application Priority Data
Jul. 31, 2013 (JP) 2013-159930

(51) Int. Cl. G06F 19/00; A61B 5/145; A61G 3/00; A61B 8/00; A61B 6/03; A61B 6/00; A61B 5/0205; A61B 5/0402

(52) U.S. Cl. CPC G06F 19/321; G06F 19/322; A61B 5/02055; A61B 5/14542; A61B 5/0402; A61B 8/565; A61B 6/032; A61B 6/563; A61G 3/001

(57) ABSTRACT

In an initial emergency response step in which a patient is transported from a site of emergency to a hospital, when a paramedic starts treatment of the patient using a medical device such as a triage device or a vital sign measurement device, treatment start information is transmitted from the medical device to a medical support server of a medical support system. According to the treatment start information, the medical support server creates timeline information for managing medical care information of the patient along a time axis and starts management of the medical care information generated in the initial emergency response step.

### CROSS-REFERENCE TO RELATED APPLICATIONS

This application is a Continuation of PCT International Application No. PCT/JP2014/070032 filed on Jul. 30, 2014, which claims priority under 35 U.S.C §119(a) to Japanese Patent Application No. 2013-159930 filed Jul. 31, 2013. The above application is hereby expressly incorporated by reference, in its entirety, into the present application.

### BACKGROUND OF THE INVENTION

1. Field of the Invention

The present invention relates to a medical support system for supporting emergency medical care.

2. Description Related to the Prior Art

Conventionally, an emergency medical system in which, when an emergency arises in which a patient requires medical care from a doctor due to sudden illness, injury, or the like, a paramedic is dispatched to a site where the patient is located, and the patient is transported from the site which is a transport source to a medical facility which is a transport destination in which medical care of the patient is performed, is provided. In the medical facility, an emergency medical team including a plurality of medical staff, such as doctors or nurses as members, is on stand-by. The medical team medically examines and treats a transported patient in cooperation with each member. Thus, in modern emergency medical care, since the medical care of the patient is performed by a medical team, it is preferable that medical care information on medical examination or treatment given to the patient can be shared among the members of the medical team.

A medical support system in which medical care information of a patient can be shared between a plurality of medical staff are described in JP2012-027565A (US Patent App. No. 2012/0022885). The medical support system includes a plurality of client terminals respectively used by a plurality of medical staff, and a medical support server that creates timeline information of a patient and distributes the timeline information to each client terminal. The timeline information is information for managing various types of medical care information regarding medical examination, inspection, treatment, or the like of the patient along a time axis. According to the medical support system, the medical care information of the patient can be shared between a plurality of medical staff by distribution of the timeline information. Further, a temporal flow regarding medical care of the patient can be easily recognized from the timeline information.

Incidentally, in emergency medical care, medical care information obtained in an initial emergency response step is known to be useful for administering medical care to the patient accurately and rapidly. The medical care information in the initial emergency response step is medical care information obtained in the initial emergency response step in which the patient is transported from a site to a medical facility and is, for example, information indicating the state of a patient at the site or in an ambulance, the content or a result of treatment by a paramedic, the state of the site, or the like. However, in the medical support system disclosed in JP2012-027565A, since the patient is transported to the medical facility, capturing of an inspection image is performed and then the timeline information is created, medical care information in the initial emergency response step is not registered in the timeline information. Therefore, the medical care information in the initial emergency response step cannot be obtained from the timeline information, and medical care cannot be administered to the patient.

The content of JP2004-280807A does not relate to timeline information and discloses a medical support system in which medical care information of patients can be shared among a plurality of doctors. This medical support system includes a patient terminal that is used by a patient, a plurality of client terminals that are respectively used by a plurality of doctors belonging to a plurality of medical facilities, and a medical support server that connects the patient terminal to the plurality of client terminals over a communication network to build a cyber hospital in response to a command from the patient terminal. The cyber hospital is a virtual hospital in which a plurality of doctors cooperate with one another over a communication network to administer medical care to patients.

In the medical support system disclosed in JP2004-280807A, when the patient cannot operate the patient terminal due to his or her sudden illness, the paramedic transporting the patient can operate a wireless terminal in the ambulance in place of the patient to instruct the medical support server to build the cyber hospital. When this technology is applied to the medical support system in JP2012-027565A, the medical support server can start the creation of the timeline information in response to an operation of the terminal by the paramedic in the initial emergency response step in which the patient is transported from a site to the medical facility. Further, since medical care information in the initial emergency response step can be registered in the created timeline information, medical care of the patient can be performed by referring to the medical care information in the initial emergency response step in the medical facility to which the patient is transported.

However, since the paramedic should preferentially perform treatment and transport of the patient to a medical facility, the paramedic may not have time to operate the terminal and instruct the medical support server to create the timeline information in an initial emergency response step. Therefore, when an operation for creating the timeline information is delegated to the paramedic, a timing at which the timeline information is created is likely to be delayed and useful medical care information in the initial emergency response step is likely not to be obtained. Further, when the paramedic is forced to perform creation of the timeline information, this is also likely to interfere with treatment of patients.

### SUMMARY OF THE INVENTION

An object of the present invention is to provide a medical support system capable of creating timeline information in an initial emergency response step without interfering with the duties of the paramedic.

In order to solve the above problems, a medical support system of the present invention includes a medical device used for treatment of patients, and a medical support server that is communicably connected to the medical device over a communication network. The medical device is used by a paramedic to treat the patient in an initial emergency response step in which the patient is transported from a site as a transport source to a medical facility as a transport destination. The medical device includes a treatment start information transmission unit for transmitting treatment start information indicating that treatment for the patient has started to the medical support server upon the medical device being used. The medical support server includes a treatment start information reception unit and a timeline creation unit. The treatment start information reception unit receives the treatment start information from a medical device. The timeline creation unit creates timeline information for managing medical care information of the patient along a time axis in the case where the treatment start information reception unit receives the treatment start information.

It is preferable that a patient ID for identifying the patient is imparted to the treatment start information. Further, it is preferable that the timeline creation unit creates one piece of timeline information for each patient ID.

It is preferable that the patient ID is a temporary ID that is temporarily issued in the initial emergency response step.

It is preferable that the medical device includes a patient ID reception unit for receiving the patient ID input manually or the patient ID transmitted from the medical support server. Further, it is preferable that the patient ID received by the patient ID reception unit is imparted to the treatment start information.

It is preferable that the treatment start information reception unit is adapted to receive the treatment start information from a plurality of the medical devices. Further, it is preferable that the timeline creation unit creates the timeline information based on initially received treatment start information among a plurality of pieces of the treatment start information including the same patient ID received by the treatment start information reception unit.

It is preferable that the treatment start information reception unit receives first medical care information acquired from the patient by the medical device, as the treatment start information.

It is preferable that the medical support server includes a medical care information registration unit for receiving the medical care information transmitted from the medical device and registering the received medical care information in the timeline information.

It is preferable that at least one of a triage device, a vital sign measurement device, and a photographing device is included in the medical device. The triage device is a device for classifying patients according to a degree of severity. The vital sign measurement device is a device for measuring vital signs of the patient. The photographing device is a device for capturing a diagnosis image of the patient.

It is preferable that the medical device is adapted to be mounted on an ambulance that transports the patient to the medical facility.

It is preferable that the medical support server includes a distribution unit for distributing the timeline information to a client terminal over a communication network.

It is preferable that the medical care information registration unit is adapted to register information transmitted from the client terminal as the medical care information in the timeline information.

It is preferable that the client terminal is used by the paramedic or doctor to input a comment to the timeline information.

It is preferable that the medical support server includes a timeline termination unit for performing a termination process of terminating an operation of the timeline information.

It is preferable that the medical support server includes a timeline transfer unit for transferring all or part of a management function of managing the timeline information to another server after the initial emergency response step has ended.

It is preferable that the timeline transfer unit transfers at least one of a storage destination for the timeline information, and a registration right for the medical care information.

It is preferable that the other server as a transport destination for the timeline information is an in-hospital server managed by the medical facility as the transport destination of the patient.

According to the present invention, since the timeline information is created with start of treatment of a patient by a paramedic, it is possible to create the timeline information in an initial emergency response step without interfering with the duties of the paramedic. Further, since the medical care information in the initial emergency response step can be registered in the created timeline information, it is possible to appropriately care the patient by referring to the medical care information in the initial emergency response step.

### BRIEF DESCRIPTION OF DRAWINGS

For more complete understanding of the present invention, and the advantage thereof, reference is now made to the subsequent descriptions taken in conjunction with the accompanying drawings, in which:

FIG. 1 is a schematic diagram of an emergency medical system;

FIG. 2 is an illustrative diagram illustrating a range of initial emergency response steps in emergency care;

FIG. 3 is a schematic diagram illustrating a configuration of a medical support system of the present invention;

FIG. 4 is a block diagram illustrating a configuration of a computer used for a medical support server;

FIG. 5 is a block diagram illustrating a functional configuration of a medical support system;

FIG. 6 is a block diagram illustrating a functional configuration of a timeline management unit;

FIG. 7 is an illustrative diagram illustrating a configuration of triage information;

FIG. 8 is an illustrative diagram illustrating a configuration of timeline information;

FIG. 9 is an illustrative diagram illustrating a configuration of a timeline screen;

FIG. 10 is an illustrative diagram illustrating a configuration of a timeline screen in which triage information is displayed;

FIG. 11 is an illustrative diagram illustrating a configuration of a timeline screen in which an ultrasonic image is displayed;

FIG. 12 is an illustrative diagram illustrating a configuration of a timeline screen in which a comment input window is displayed;

FIG. 13 is an illustrative diagram illustrating a configuration of a timeline screen in which an operation termination registration window is displayed;

FIG. 14 is an illustrative diagram illustrating an operation termination screen of the timeline information;

FIG. 15 is a flowchart illustrating a procedure of distributing a temporary patient ID;

FIG. 16 is a flowchart illustrating a procedure of creating the timeline information;

FIG. 17 is a flowchart illustrating an operation termination procedure for the timeline information;

FIG. 18 is a block diagram illustrating a configuration of an emergency portable terminal having a function of inputting a patient ID;

FIG. 19 is a block diagram illustrating a configuration of a medical support server of a second embodiment;

FIG. 20 is a flowchart illustrating a procedure of transferring a function of managing timeline information; and

FIG. 21 is a conceptual diagram regarding transfer of the function of managing timeline information.

### DESCRIPTION OF THE PREFERRED EMBODIMENTS

### First Embodiment

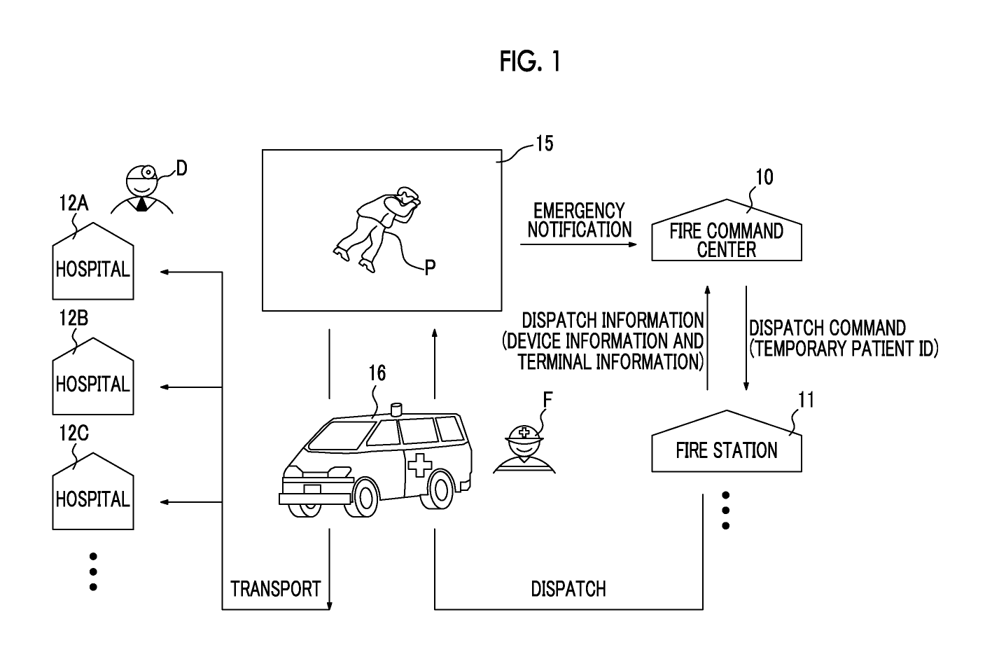

An emergency medical system in which, when an emergency patient requiring medical care of a doctor is generated due to sudden illness, injury, or the like, a paramedic is dispatched to a site where the patient waits, and the patient is transported from the site to a medical facility in which medical care of the patient is performed, is provided in each municipality of an administrative district or the like. As illustrated in FIG. 1, this emergency medical system is built by, for example, a fire command center 10 that is a facility playing a central role in the emergency medical care in the area, a plurality of fire stations 11, and a plurality of hospitals that are medical facilities, such as a hospital 12A, a hospital 12B, and a hospital 12C.

The fire command center 10 receives an emergency notification from the patient P who is at a site 15, specifies an address of the site 15 from notification content, and instructs the fire station 11 close to the site 15 to dispatch paramedics. The fire station 11 organizes the paramedics consisting of a plurality of paramedics F, including emergency response personnel, and dispatches an ambulance 16 in response to the dispatch command. After arriving at the site 15, the paramedics perform emergency treatment on the patient P, place the patient P in the ambulance 16, and transport the patient P to any one of the hospitals 12A to 12C. The emergency response personnel is a paramedic who has acquired an emergency response personnel qualification, and can perform ensuring of an intravenous line, tracheal intubation, drug administration, and treatment using a semi-automatic defibrillator on a patient in a state of cardiopulmonary arrest under instructions of a doctor.

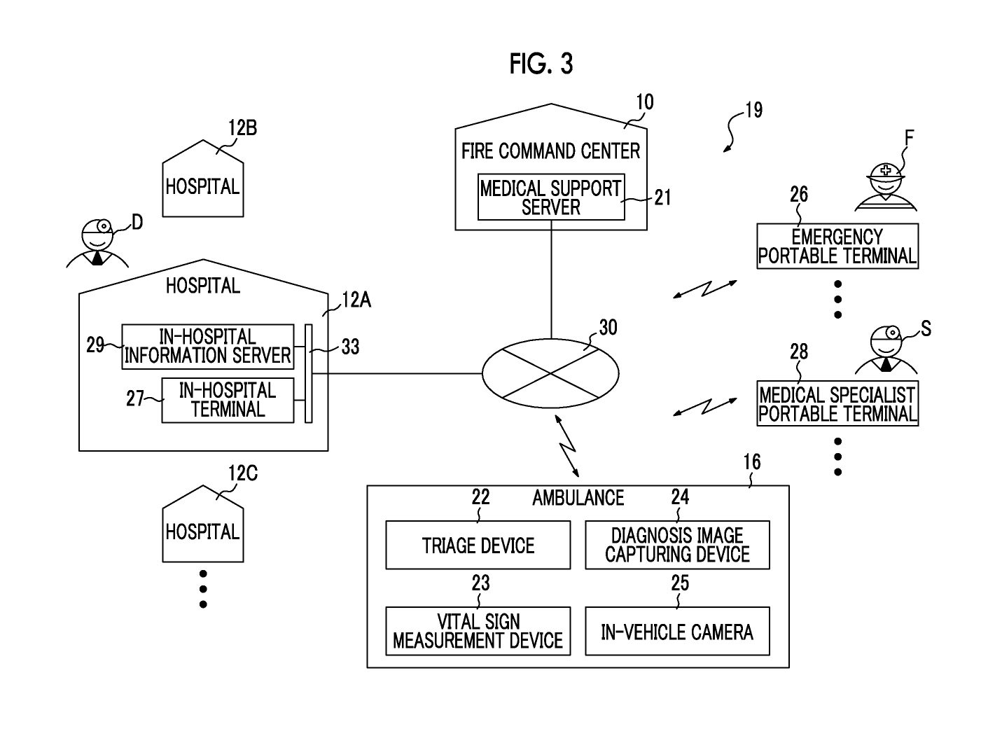

The hospitals 12A to 12C are emergency designated hospitals including an emergency medical facility, and an emergency medical team including a plurality of medical staff such as doctors D or nurses as members. The emergency medical team medically examines and treats the transported patient P in cooperation with each member. Further, in the emergency medical system, medical specialists S (see FIG. 3) with expert knowledge of a disease in a specific field may be added, in order to deal with diseases in specific fields that are difficult for the emergency medical team to diagnose.

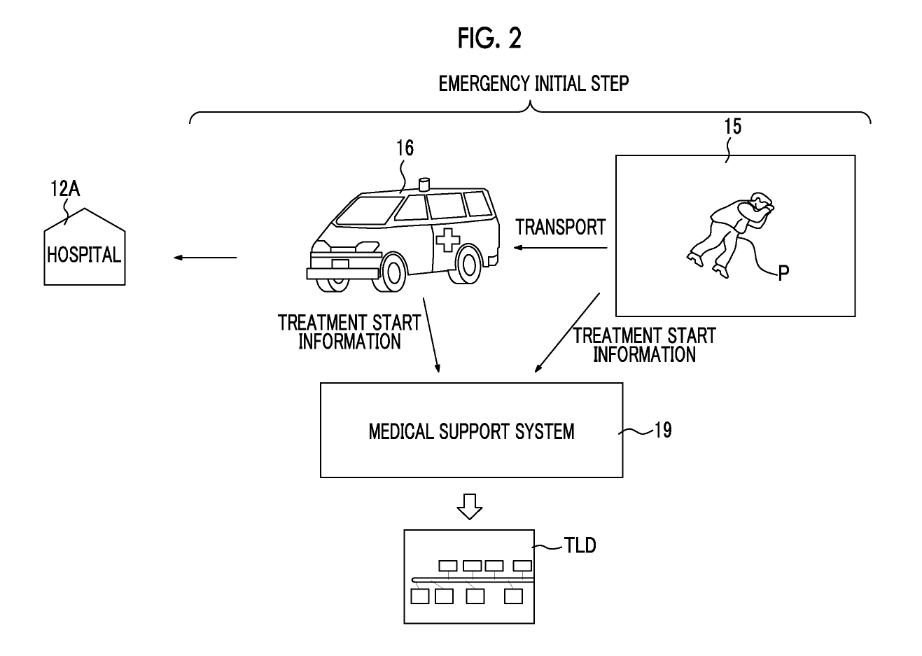

As illustrated in FIG. 2, a medical support system 19 of the present invention is a computer system that creates timeline information TLD of the patient P according to treatment start information indicating start of treatment of the patient P by paramedics in an initial emergency response step in which the patient P is transported from the site 15 which is a transport source to anyone of the hospitals 12A to 12C which is a transport destination by the ambulance 16. The timeline information is information for managing medical care information of the patient P along a time axis. Further, the medical care information is information on medical examination, inspection, or treatment given to the patient P, and includes varying image information, such as inspection images. Since the medical support system 19 creates the timeline information TLD for each patient, when a plurality of patients are generated due to accident or disaster, a plurality of pieces of timeline information TLD are created for each patient. The medical support system 19 distributes the created timeline information TLD to the client terminal used by a doctor D or the like.

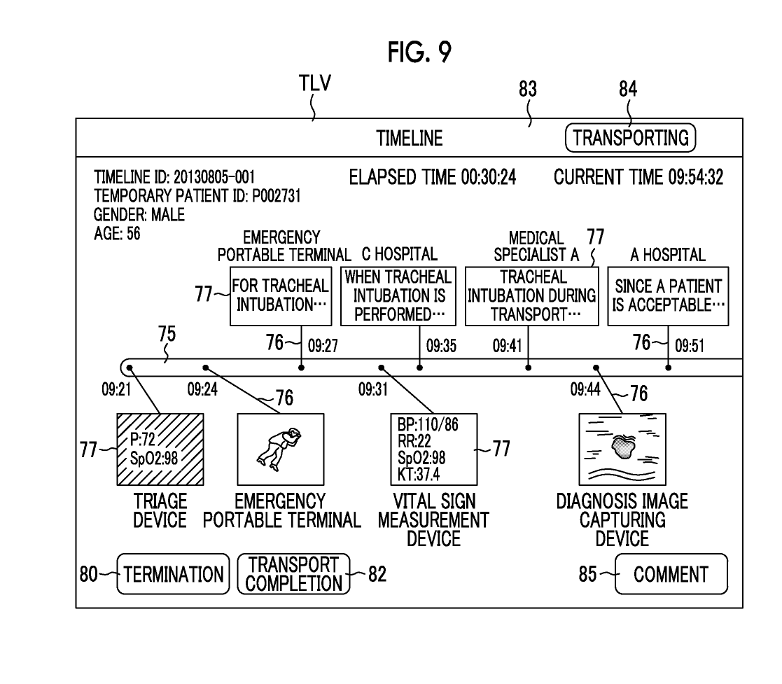

The timeline information TLD distributed to the client terminal is displayed as a timeline screen TLV on the display of the client terminal (see FIG. 9). In the timeline screen TLV, a strip time bar 75 indicating the lapse of time from medical care start for the patient P is displayed in a horizontal direction. In the time bar 75, the time regresses to the left of the timeline screen TLV, and arrives at a creation time of the timeline information TLD. Further, in the time bar 75, the time progresses to the right of the timeline screen TLV, and arrives at a current time.

A plurality of information display frames 77 are arranged along the time bar 75 over and under the time bar 75. Various medical care information on medical care given to the patient P is displayed in the information display frame 77. Each information display frame 77 and the time bar 75 are connected to each other by a lead line 76. Since each lead line 76 is connected to a position on the time bar 75 corresponding to an occurrence time of the medical care information of the information display frame 77 to which the lead line 76 is connected, the temporal progress of the medical care can be readily understood.

As illustrated in FIG. 3, the medical support system 19 includes, for example, a medical support server 21, a triage device 22, a vital sign measurement device 23, a diagnosis image capturing device 24, an in-vehicle camera 25, an emergency portable terminal 26, an in-hospital terminal 27, a medical specialist portable terminal 28, and an in-hospital information server 29. Each device constituting the medical support system 19 is connected by a communication network 30. The communication network 30 includes the Internet or a mobile communication network.

The medical support server 21 is installed in, for example, the fire command center 10. The medical support server 21 creates timeline information TLD of the patient P when receiving the treatment start information indicating that treatment of the patient P has started in an initial emergency response step. The medical support server 21 distributes the created timeline information TLD to the emergency portable terminal 26, the in-hospital terminal 27, and the medical specialist portable terminal 28, which are client terminals.

The medical support server 21 receives medical care information of the patient P generated after the creation of the timeline information TLD, and registers the received medical care information in the timeline information TLD. Further, when the patient P is transported to one of the hospitals 12A to 12C, the medical support server 21 requests the in-hospital information server 29 of the hospital to which the patient P has been transported to distribute the medical care information of the patient P, and registers the medical care information distributed from the in-hospital information server 29 in the timeline information TLD in response to the request. The timeline information TLD updated by the registration of the medical care information is re-distributed to the client terminals 26 to 28.

The triage device 22, the vital sign measurement device 23, the diagnosis image capturing device 24, and the in-vehicle camera 25 are medical devices used by the paramedic F in order to treat the patient P, and are mounted on the ambulance 16. The triage device 22, the vital sign measurement device 23, the diagnosis image capturing device 24, and the in-vehicle camera 25 are used for treatment of the patient P by the paramedic F to generate the medical care information of the patient P and transmit the generated medical care information to the medical support server 21. The medical support server 21 identifies the medical care information that is initially received among the plurality of pieces of medical care information transmitted from the triage device 22, the vital sign measurement device 23, the diagnosis image capturing device 24, and the in-vehicle camera 25, as the treatment start information described above, and starts creation of the timeline information TLD.

The triage device 22 is a device that displays a degree of severity indicating urgency of the medical care for the patient P. The triage device 22 is mounted within the ambulance 16, or on the body of the patient P at the site 15 brought out from the ambulance 16. Conventionally, triage work of classifying patients based on a degree of severity and displaying the degree of severity using a triage tag attached to the body of the patient in order to determine priority of care when a large number of patients are generated in large-scale disaster, is known. The triage device 22 is used in place of a conventional triage tag, and includes a setting function of setting the degree of severity, a function of displaying the set degree of severity, and a transmission function of transmitting triage information including the set degree of severity as medical care information of the patient P to the medical support server 21.

The vital sign measurement device 23 is a bio-monitor which is installed in the ambulance 16, and measures vital signs such as blood pressure, breathing rate, blood oxygen saturation level, body temperature, and an electrocardiogram of the patient P carried into the ambulance 16. The vital sign measurement device 23 transmits vital information including measurement results of the vital signs as the medical care information of the patient P to the medical support server 21.

The diagnosis image capturing device 24 is, for example, an ultrasonic imaging device which is installed in the ambulance 16, and captures an ultrasonic image of the patient P carried into the ambulance 16. The diagnosis image capturing device 24 transmits ultrasonic image data of the patient P to the medical support server 21 as medical care information of the patient P. In addition to the ultrasonic imaging device, an X-ray imaging device, a computed tomography (CT) imaging device, or the like may be used as the diagnosis image capturing device 24.

The in-vehicle camera 25 is installed on, for example, a ceiling of the ambulance 16, and captures a still image or a moving image of the patient P carried into the ambulance 16 so that the image is provided for diagnosis of the patient P. Therefore, the in-vehicle camera 25 is included in the diagnosis image capturing device which is one medical device of the present invention. The in-vehicle camera 25 transmits in-vehicle imaging data including data of a still image or a moving image of the patient P as medical care information of the patient P to the medical support server 21.

The emergency portable terminal 26 is a portable terminal carried by the paramedic F organized to be included in paramedics at the time of dispatching. A so-called smart phone is used. The emergency portable terminal 26 is used for, for example, inquiry about advice for emergency treatment, or inquiry about whether or not a patient is allowed to be transported to the hospitals 12A to 12C based on a phone function of the smart phone. Further, the emergency portable terminal 26 is also used for, for example, capturing of a still image or a moving image of the site 15 or the patient P, and recording of interview with the patient P or a witness through a photographing function and a recording function of the smart phone. Portable imaging data including a still image, a moving image, or audio data is transmitted to the medical support server 21 as the medical care information of the patient P.

The emergency portable terminal 26 is also used for viewing of the timeline information TLD distributed from the medical support server 21, and inputting of a comment for the timeline information TLD. For the input of the comment, a text input function of the smart phone is used. The comment input to the emergency portable terminal 26 is transmitted to the medical support server 21 as the medical care information of the patient P. Further, when transport of the patient P to the hospital is complete, the emergency portable terminal 26 transmits transport completion information indicating that the transport of the patient P to the hospital has been completed, to the medical support server 21.

In the hospitals 12A to 12C, respective hospital information systems that manage various types of medical care information in the hospitals are provided. The hospital information system includes an in-hospital information server 29, at least one in-hospital terminal 27, and a hospital network 33 that connects the in-hospital information server 29 to the in-hospital terminal 27. The hospital network 33 is built as an Intranet using the same communication protocol as that of the Internet, and is connected to the communication network 30 via a firewall (not illustrated).

A hospital information system of the hospital 12A will be described. The in-hospital information server 29 is a computer system that manages electronic medical chart information and a diagnosis image of the patient P. The electronic medical chart information is stored in an electronic medical chart database (not illustrated; a database is hereinafter referred to as a DB) for each patient. The electronic medical chart information includes, for example, personal information and medical care information of the patient P. The personal information includes, for example, patient name, in-hospital patient ID, date of birth, gender, and address. The medical care information is information on medical examination, inspection, and treatment performed on the patient P in the hospital, and includes, for example, medical care date, medical department, injury name, diagnosis result, a type and an amount of medication, and a prescription pharmacy name. The in-hospital information server 29 receives the electronic medical chart information created by the in-hospital terminal 27, and stores the electronic medical chart information in the electronic medical chart database. Further, the in-hospital information server 29 searches for the electronic medical chart DB in response to the distribution request from the in-hospital terminal 27 or the medical support server 21, and distributes the searched electronic medical chart information to the in-hospital terminal 27 or the medical support server 21.

A diagnosis image capturing device such as an X-ray imaging device, a CT imaging device, a magnetic resonance imaging (MRI) imaging device, an ultrasonic imaging device, and an endoscope is connected to the in-hospital information server 29. The in-hospital information server 29 receives a diagnosis image captured by the diagnosis image capturing device, and stores the diagnosis image in a diagnosis image DB (not illustrated). The diagnosis image stored in the diagnosis image DB is associated with an in-hospital patient ID such that a patient corresponding to the diagnosis image can be identified. Further, the in-hospital information server 29 searches for the diagnosis image DB in response to a distribution request from the in-hospital terminal 27 or the medical support server 21, and distributes the searched diagnosis image to the in-hospital terminal 27 or the medical support server 21.

The in-hospital terminal 27 is a client terminal that is installed in each medical department in a hospital, and is operated by a doctor D or a nurse in each medical department. The in-hospital terminal 27 is used, for example, for input of the electronic medical chart information to the in-hospital information server 29, viewing of the electronic medical chart information and the diagnosis image, and reservation of imaging of the diagnosis image for the diagnosis image capturing device.

The in-hospital terminal 27 is also used for viewing of the timeline information TLD distributed from the medical support server 21, and input of a comment to the timeline information TLD. A comment input to the in-hospital terminal 27 is transmitted to the medical support server 21 as the medical care information of the patient P. Since the in-hospital information server and the in-hospital terminal provided in the hospitals 12B and 12C have the same configuration as that of the in-hospital information server 29 and the in-hospital terminal 27 in the hospital 12A, detailed description thereof will be omitted.

The medical specialist portable terminal 28 is a portable terminal that is carried by a medical specialist S which is registered in the medical support server 21 in advance. A so-called smart phone is used. The specialist S is a doctor with expert knowledge of the specific field, such as cerebrovascular disease or cardiovascular disease, and provides, for example, advice about treatment or an opinion regarding medical care for a critically ill patient for the paramedic F, the doctor D of the hospital, or the like. The medical specialist portable terminal 28 is also used for viewing of the timeline information TLD distributed from the medical support server 21, and input of a comment to the timeline information TLD. Advice regarding treatment or an opinion regarding the medical care of medical specialist S is provided by the comment on the timeline information TLD. A comment input to the medical specialist portable terminal 28 is transmitted to the medical support server 21 as the medical care information of the patient P.

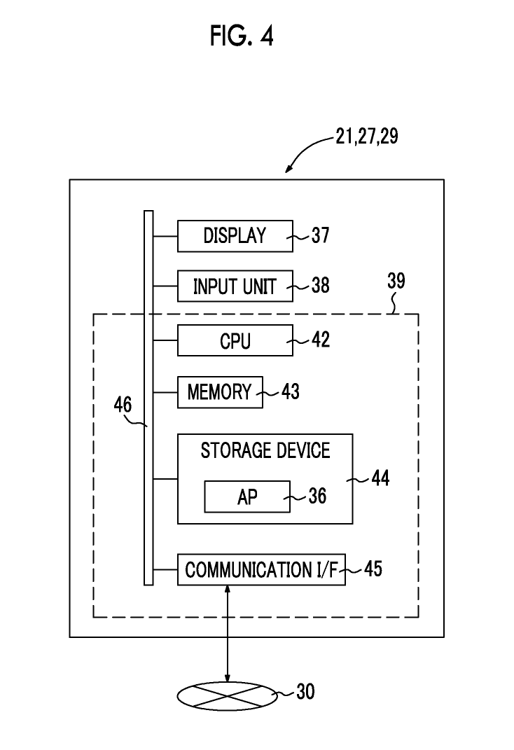

As illustrated in FIG. 4, the medical support server 21, the in-hospital terminal 27, and the in-hospital information server 29 are constituted by a computer such as a personal computer or a workstation. The computer operates a control program such an operating system or an application program (AP) 36 for causing the computer to function as a desired server or terminal, so as to function as the medical support server 21, the in-hospital terminal 27, and the in-hospital information server 29. The computer constituting the medical support server 21, the in-hospital terminal 27, and the in-hospital information server 29 includes a display 37, an input unit 38 such as a keyboard or a mouse, and a computer main body 39.

A central processing unit (CPU) 42, a memory 43, a storage device 44, and a communication I/F 45 are provided in the computer main body 39, and are connected to each other via a data bus 46. The storage device 44 is a device that stores various data and includes, for example, a hard disk drive. The AP 36 such as a control program, a medical support program, and various databases is stored in the storage device 44.

The memory 43 is a work memory used for the CPU 42 to execute a process. The CPU 42 generally controls each unit of the computer by loading the control program stored in the storage device 44 into the memory 43 and executing the process according to the program. The communication I/F 45 is a communication interface for connecting to the communication network 30 and communicating with each device of the medical support system 19.

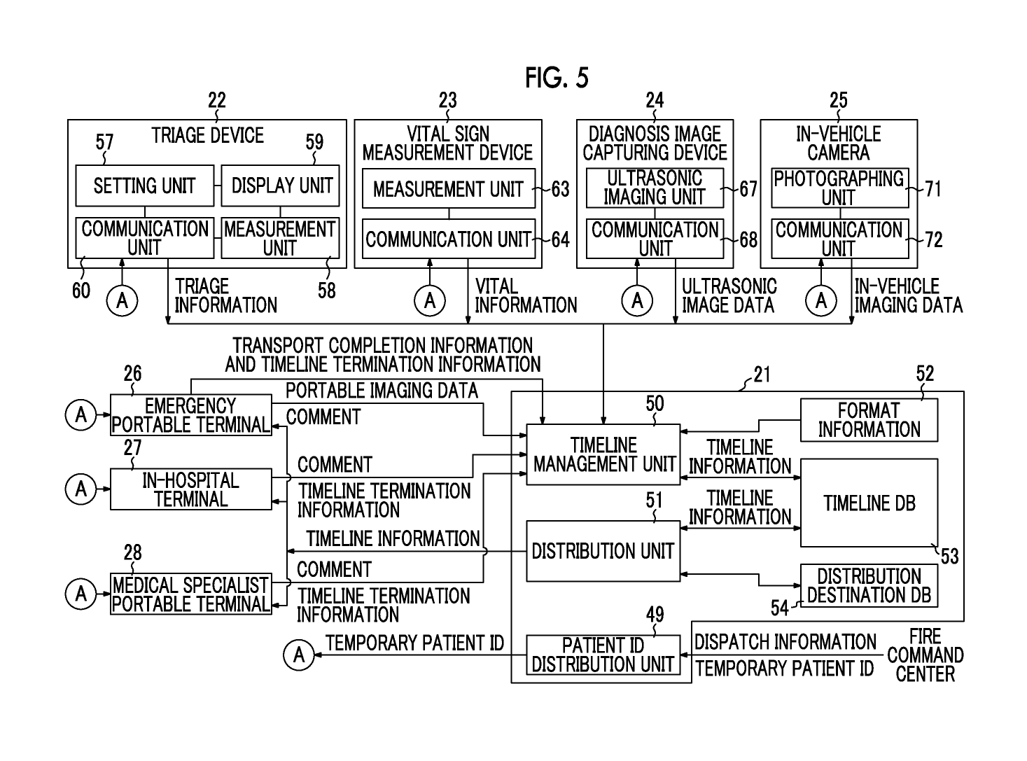

As illustrated in FIG. 5, when the medical support program starts up, the CPU 42 of the medical support server 21 functions as a patient ID distribution unit 49, a timeline management unit 50, and a distribution unit 51 in cooperation with the memory 43. Further, format information 52 of the timeline information TLD, a timeline DB 53 in which created timeline information TLD is stored, and a distribution destination DB 54 in which distribution information on a client terminal to which the timeline information TLD is distributed is stored in the storage device 44 of the medical support server 21.

The patient ID distribution unit 49 distributes the temporary patient ID of the patient P to the respective medical devices 22 to 25 and the respective client terminals 26 to 28. The temporary patient ID distributed from the patient ID distribution unit 49 is transmitted together with the medical care information when the medical care information of the patient is transmitted from each of the medical devices 22 to 25 and each of the client terminals 26 to 28 to the medical support server 21.

In the medical support system 19, for example, when a large number of patients are generated by accident, disaster, or the like, a plurality of pieces of timeline information TLD are created for each patient, and accordingly, it is necessary for a patient corresponding to medical care information transmitted from each of the medical devices 22 to 25 and each of the client terminals 26 to 28 to be identified. Therefore, the patient ID distribution unit 49 distributes the temporary patient ID for identifying the medical care information to the medical device and the client terminal, and the medical device and the client terminal transmits the medical care information together with the temporary patient ID when transmitting the medical care information to the medical support server 21.

The distribution of the temporary patient ID is performed, for example, in the following procedure. A fire emergency system (not illustrated) that performs a dispatch command or the like in the fire command center 10, imparts a temporary patient ID to the patient P according to emergency notification, and transmits a dispatch command including the temporary patient ID to the fire station 11. A dispatch processing system (not illustrated) that processes the dispatch command in the fire station 11 determines the ambulance 16 to be dispatched after paramedics are organized, and transmits dispatch information including device information of each of the medical devices 22 to 25 mounted on the ambulance 16, terminal information of the emergency portable terminal 26 of each paramedic F to be dispatched, and the like to the fire emergency system. The fire emergency system receiving the dispatch information from the dispatch processing system transmits the temporary patient ID and the dispatch information corresponding to the temporary patient ID to the medical support server 21 over the communication network 30.

The device IDs of the medical devices 22 to 25 and communication information necessary for communication with the medical devices 22 to 25 are included in the device information in the dispatch information described above. Further, the terminal ID of the emergency portable terminal 26 and communication information necessary for communication with the emergency portable terminal 26 are included in the terminal information of the dispatch information. For example, an IP address or a MAC address imparted to each of the medical devices 22 to 25 and the emergency portable terminal 26 is included in the communication information of the medical devices 22 to 25 and the emergency portable terminal 26. When the patient ID and the dispatched information are transmitted from the fire emergency system to the medical support server 21, emergency notification reception time, gender and age or year of patient P may be transmitted together.

The patient ID distribution unit 49 distributes the temporary patient ID to each of the medical devices 22 to 25 and each emergency portable terminal 26 based on the communication information included in the dispatch information. Further, the patient ID distribution unit 49 also distributes the temporary patient ID to each in-hospital terminal 27 and each medical specialist portable terminal 28 registered in the distribution destination DB 54. Each of the medical devices 22 to 25 and each of the client terminals 26 to 28 receiving the temporary patient ID impart the temporary patient ID to the medical care information when transmitting the medical care information to the medical support server 21. Accordingly, the medical support server 21 can identify a patient corresponding to the received medical care information based on the temporary patient ID.

The in-hospital information server 29 of the hospital to which the patient P has been transported imparts the in-hospital patient ID used in the hospital to the patient P, and transmits the in-hospital patient ID to the medical support server 21. The medical support server 21 registers the in-hospital patient ID to the timeline information TLD of the patient P, and manages the timeline information TLD of the patient P based on the in-hospital patient ID after transport to the hospital.

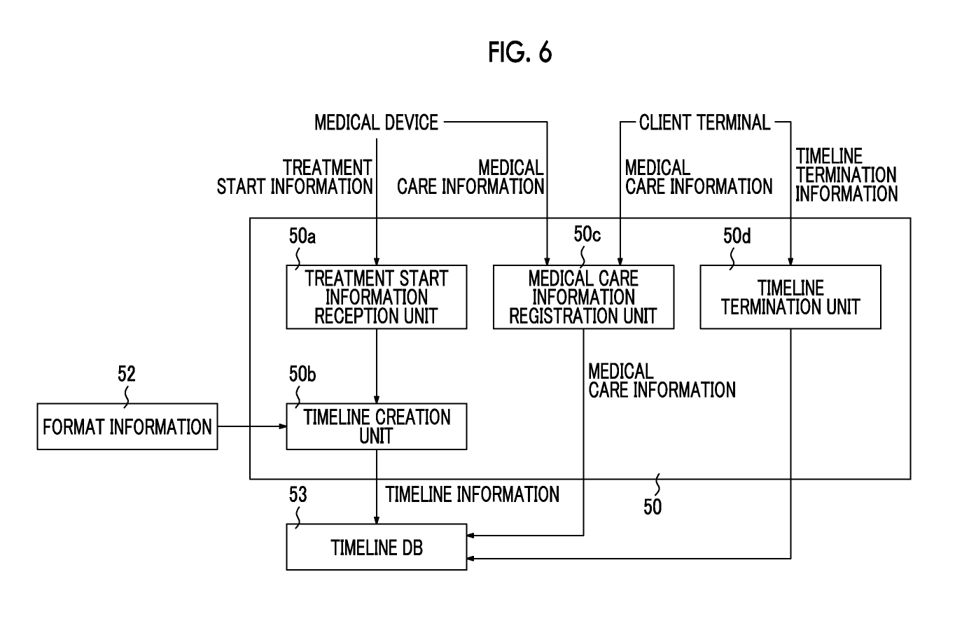

As illustrated in FIG. 6, the timeline management unit 50 functions as a treatment start information reception unit 50a that receives the treatment start information from each of the medical devices 22 to 25, a timeline creation unit 50b that creates the timeline information TLD when receiving the treatment start information, and a medical care information registration unit 50c that receives medical care information transmitted from each of the medical devices 22 to 25 and each of the client terminals 26 to 28 and registers the received medical care information in the timeline information TLD. Further, the timeline management unit 50 also functions as a timeline termination unit 50d that terminates an operation of the created timeline information TLD.

The treatment start information reception unit 50a receives, as the treatment start information, first medical care information that is acquired from the patient P by the medical device such as the triage device 22, the vital sign measurement device 23, the diagnosis image capturing device 24, and the in-vehicle camera 25. Therefore, even when the timeline management unit 50 receives a plurality of pieces of medical care information from the respective medical devices 22 to 25, the treatment start information reception unit 50a does not receive, as treatment start information, medical care information received at second or a subsequent time.

When receiving the medical care information to be handled as the treatment start information, the timeline creation unit 50b creates one piece of timeline information TLD for each temporary patient ID imparted to the received medical care information. Therefore, when a plurality of pieces of treatment start information with different temporary patient IDs are received, timeline information TLD corresponding to each temporary patient ID is created. Accordingly, the same number of timeline information TLD as the number of patients is created, and accordingly, it is possible to prevent mismanagement such as medical care information of one patient being managed in a plurality of timelines data TLD, and mismanagement such as medical care information of a plurality of patients being managed in one piece of timelines data TLD.

The timeline creation unit 50b registers the medical care information received as the treatment start information and the temporary patient ID in the format information 52 read from the storage device 44 of the medical support server 21, to create the timeline information TLD. The timeline creation unit 50b imparts a timeline ID to the created timeline information TLD and stores the resultant timeline information TLD in the timeline DB 53.

When the medical care information is received from each of the medical devices 22 to 25 and each of the client terminals 26 to 28 after the timeline information TLD is created, the medical care information registration unit 50c confirms the temporary patient ID in the received medical care information, and registers the received medical care information in the timeline information TLD corresponding to the temporary patient ID. Further, when the patient P is transported to the hospital, the medical care information registration unit 50c designates the in-hospital patient ID of the patient P, requests the in-hospital information server 29 of the hospital to which the patient P has been transported to distribute the medical care information and the diagnosis image, and registers, in the timeline information TLD, the medical care information and the diagnosis image distributed from the in-hospital information server 29.

The timeline termination unit 50d terminates the operation of the timeline information TLD when the timeline information TLD is not required due to a condition of the patient being mild, or when the timeline information TLD is no longer required due to patient's recovery, death, or the like. The timeline termination unit 50d performs a termination process when a terminating operation is performed in the medical support server 21, or when timeline termination information is transmitted from the client terminal, such as the emergency portable terminal 26, the in-hospital terminal 27, and the medical specialist portable terminal 28. In this termination process, “operation termination” and a reason for operation termination are registered as operation status indicating operational situation of the timeline information in the timeline information TLD of the patient that is a termination target. For the timeline information TLD in which the operation status is “operation termination”, the registration of the medical care information is then completed and an operation termination screen is distributed to the client terminals 26 to 28.

The distribution unit 51 reads the timeline information TLD stored in the timeline DB 53, and creates the timeline screen TLV based on the read timeline information TLD. Further, the distribution unit 51 distributes the timeline screen TLV to the client terminals 26 to 28 based on the distribution information stored in the distribution destination DB 54. Communication information required to distribute the timeline information TLD to each emergency portable terminal 26, each in-hospital terminal 27, and each medical specialist portable terminal 28 is registered in the distribution information. Since treatment of an initial emergency response step for the patient P terminates after transport of the patient P to the hospital, the distribution unit 51 terminates the distribution of medical care information to the emergency portable terminal 26 after the transport of the patient P to the hospital. Further, when the operation of the timeline information TLD terminates in the timeline termination unit 50d, the distribution unit 51 distributes the operation termination screen to the client terminals 26 and 27.

The triage device 22 includes a setting unit 57 that sets a degree of severity of the patient P, a measurement unit 58 that measures vital signs of the patient P, a display unit 59 that displays the degree of severity and a result of the measurement, and a communication unit 60 that creates triage information including the degree of severity and the measurement result and transmits the triage information to the medical support server 21 as the medical care information of the patient P.

The setting unit 57 and the display unit 59 are constituted by, for example, a touch panel using a color liquid crystal panel. The paramedic F generally determines the degree of severity of the patient P using a determination method called the START method, and operates the touch panel to set the degree of severity. In the START method, the degree of severity of the patient is classified into four steps: “deceased, or life-saving is impossible”, “in a serious life-threatening state”, “not in a serious life-threatening state, but transport is necessary”, and “emergency transport is not necessary” based on whether a patient can walk, a breathing state, a breathing rate, a circulation state, and a level of consciousness. Four identification colors including black, red, yellow, and green are assigned to each degree of severity. An identification mark of the identification color corresponding to the degree of severity set by the setting unit 57 is displayed on the touch panel.

The measurement unit 58 has the same function as a pulse oximeter that is conventionally used in the medical field. The measurement unit 58 irradiates the body such as an ear or a finger tip of the patient P with red light and infrared light from a light emitting unit built into a mounting unit mounted on a body such as an ear or a fingertip of the patient P, and receives light transmitted through or reflected by the body of the patient P using a light reception unit built into the mounting unit. The measurement unit 58 measures a pulse and a blood oxygen saturation level based on an amount of the light received by the light reception unit.

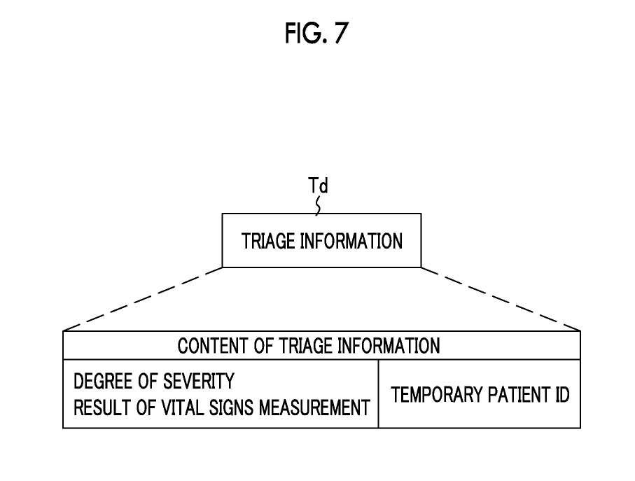

The communication unit 60 functions as the patient ID reception unit of the present invention, and receives the temporary patient ID from the patient ID distribution unit 49 of the medical support server 21. Further, the communication unit 60 functions as a treatment start information transmission unit which transmits the treatment start information indicating that the treatment for the patient P has started to the medical support server 21. As illustrated in FIG. 7, the communication unit 60 creates triage information Td as the medical care information also handled as the treatment start information. The degree of severity set by the setting unit 57, and the measurement result of the vital signs measured by the measurement unit 58 are included in the triage information Td. Further, the temporary patient ID received from the patient ID distribution unit 49 is added to the triage information Td.

The vital sign measurement device 23 includes a measurement unit 63 that measures vital signs such as blood pressure, breathing rate, blood oxygen saturation level, body temperature, and electrocardiogram of the patient P, and a communication unit 64 that transmits the vital information including measurement results of the vital signs to the medical support server 21 as the medical care information of the patient P. The communication unit 64 also functions as a patient ID reception unit and a treatment start information transmission unit, similar to the communication unit 60 of the triage device 22. Further, the temporary patient ID is added to the vital information, similar to the triage information Td.

The diagnosis image capturing device 24 includes an ultrasonic imaging unit 67 that captures an ultrasonic image of the patient P, and a communication unit 68 that transmits ultrasonic image data of the patient P as the medical care information of the patient P to the medical support server 21. The communication unit 68 also functions as a patient ID reception unit and a treatment start information transmission unit, similar to the communication unit 60 of the triage device 22. Further, the temporary patient ID is added to the ultrasonic image data, similar to the triage information Td.

The in-vehicle camera 25 includes a photographing unit 71 that captures a still image or a moving image of the patient P carried into the ambulance 16, and a communication unit 72 that transmits in-vehicle imaging data including data of the still image or the moving image of the patient P as the medical care information of the patient P to the medical support server 21. The communication unit 72 also functions as a patient ID reception unit and a treatment start information transmission unit, similar to the communication unit 60 of the triage device 22. Further, the temporary patient ID is added to the in-vehicle imaging data, similar to the triage information Td.

The temporary patient ID is imparted to the portable imaging data and the comment transmitted as the medical care information from the emergency portable terminal 26, the in-hospital terminal 27, and the medical specialist portable terminal 28 to the medical support server 21. Therefore, the medical support server 21 can specify the patient corresponding to the comment by confirming the temporary patient ID.

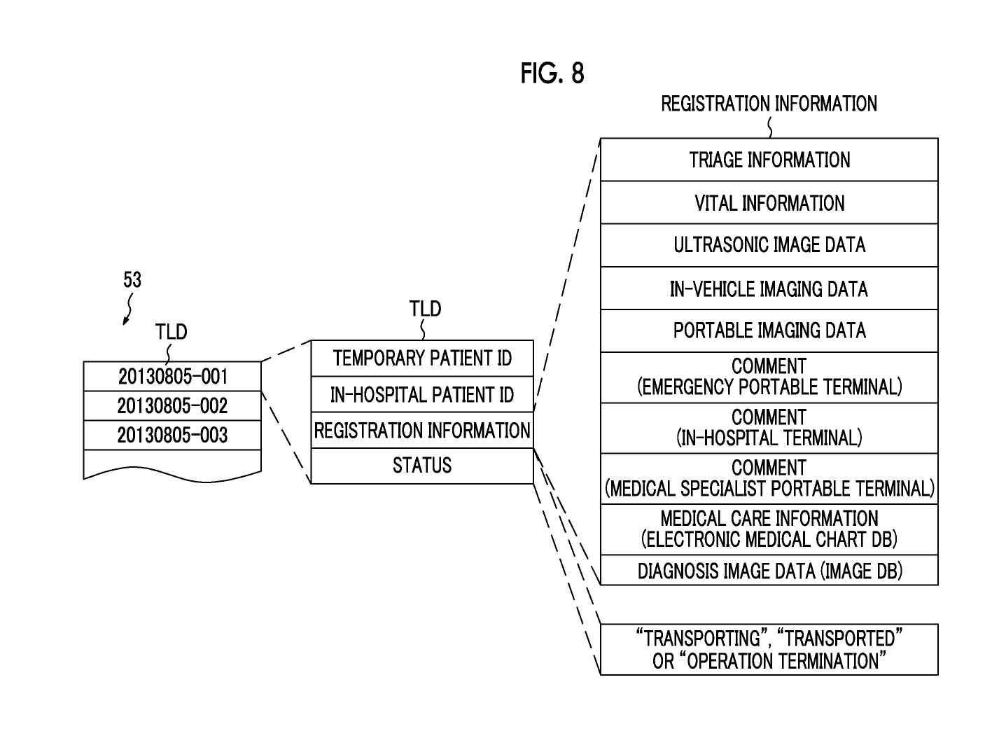

As illustrated in FIG. 8, a plurality of pieces of timeline information TLD to which the timeline ID is imparted are stored in the timeline DB 53. The timeline ID includes, for example, the date on which the timeline information TLD has been created, and identification information for identifying the plurality of pieces of timeline information TLD created on the creation date. For example, the timeline ID “20130805-001” indicates that the timeline information TLD was initially created on Aug. 5, 2013.

The timeline information TLD includes the temporary patient ID, the in-hospital patient ID, the registration information, and the status. Medical care information registered by the timeline management unit 50 is stored in the registration information. Specifically, triage information, vital information, ultrasonic image data, and in-vehicle imaging data received from the triage device 22, the vital sign measurement device 23, the diagnosis image capturing device 24, and the in-vehicle camera 25, respectively, are stored in the registration information. Further, the portable imaging data and the comment received from each emergency portable terminal 26, and the comment received from each of the in-hospital terminal 27 and the medical specialist portable terminal 28 are stored in the registration information. Furthermore, after the patient P is carried into the hospital, the medical care information of the patient P read from the electronic medical chart DB of the in-hospital information server 29, and the data of the diagnosis image read from the diagnosis image DB are also stored in the registration information.

“Transporting” indicating that the patient P is being transported to the hospital, “transported” indicating that the transport to the hospital has been completed, and “operation termination” indicating that the operation of the timeline information TLD has terminated are registered in the status. The timeline creation unit 50b registers “transporting” in the status at the time of creating the timeline information TLD. The medical care information registration unit 50c registers “transported” in the status when receiving the transport completion information from the emergency portable terminal 26. The timeline termination unit 50d registers “operation termination” in the status when a terminating operation is performed in the medical support server 21 or when timeline termination information is received from the client terminal, such as the emergency portable terminal 26, the in-hospital terminal 27, and the medical specialist portable terminal 28.

As illustrated in FIG. 9, the timeline screen TLV created based on the timeline information TLD is distributed to each of client terminals 26 to 28, and displayed on the display of each of the client terminals 26 to 28. The timeline screen TLV distributed to the emergency portable terminal 26 or the medical specialist portable terminal 28 is operated by a touch operation in the touch panel. Further, the timeline screen TLV distributed to the in-hospital terminal 27 is operated by the keyboard or the mouse.

The timeline screen TLV includes the time bar 75 indicating a flow of time in a direction from the left to the right, a plurality of the lead lines 76 extending in a vertical direction from the time bar 75, and a plurality of information display frames 77 having rectangular frame shapes at tips of the lead lines 76. A portion or a minified image of the medical care information is displayed within each information display frame 77, and a name of a transmission source of the medical care information such as a name of a medical device or a hospital is displayed over or under the information display frame 77. A transmission time of each piece of medical care information is displayed next to the lead line 76 over or under the time bar 75. When the information display frame 77 is selected in the timeline screen TLV, the medical care information corresponding to the selected information display frame 77 is read from the timeline information TLD and displayed on the timeline screen TIN.

The timeline ID, temporary patient ID, and gender and age of the patient P included in the dispatch information received from the fire emergency system are displayed to the upper left of the timeline screen TIN. The time which has elapsed since the creation of the timeline information TLD, and the current time are displayed to the upper center and to the right of the timeline screen TLV.

An termination button 80 to be operated to terminate the operation of the timeline information TLD when the timeline information TLD is not required, due to a condition of the patient being mild or when the timeline information TLD is not required due to patient's recovery, death, or the like is provided to the lower left of the timeline screen TLV. When the termination button 80 is operated, an operation termination registration screen for inputting reasons for termination and registering the operation termination is displayed on the timeline screen TIN.

A transport completion button 82 arranged to the right of the termination button 80 is operated by the paramedic F after the transport to the hospital of the patient P has been completed. The transport completion button 82 is displayed only in the timeline screen TLV of the emergency portable terminal 26 such that the inadvertent operation of a person other than the paramedic F is prevented. When the transport completion button 82 is operated, the emergency portable terminal 26 transmits transport completion information to the medical support server 21.

A transport situation display portion 84 in which a transport status of the patient P is displayed is provided to the right in a title portion 83 provided in an upper portion of the timeline screen TLV. The timeline management unit 50 displays “transporting” in the transport situation display portion 84 at the time of creation of the timeline information TLD, and changes the transport situation display portion 84 into “transported” when the transport complete information is input from the emergency portable terminal 26. After the input of the transport completion information, the timeline management unit 50 requests the in-hospital information server 29 of the hospital to which the patient P has been transported, to distribute the medical care information and the diagnosis image of the patient P, and registers, in the timeline information TLD, the medical care information and the diagnosis image distributed from the in-hospital information server 29.

A comment button 85 to be operated when a comment is input to the timeline information TLD is provided to the lower right of the timeline screen TLV. When the comment button 85 is operated, a comment input window for inputting the comment is displayed on the timeline screen TLV.

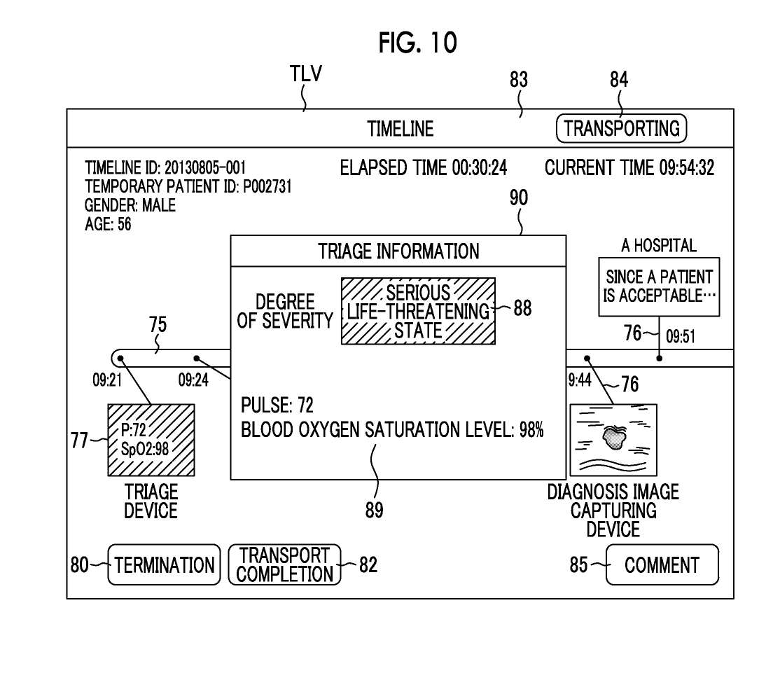

FIG. 10 illustrates a case in which the information display frame 77 of the triage device is selected in the timeline screen TLV. A sub-window 90 in which a description sentence 88 of the degree of severity in which a background color is the identification color indicating the degree of severity, and a measurement result 89 of a pulse and a blood oxygen saturation level are displayed is displayed on the timeline screen TLV.

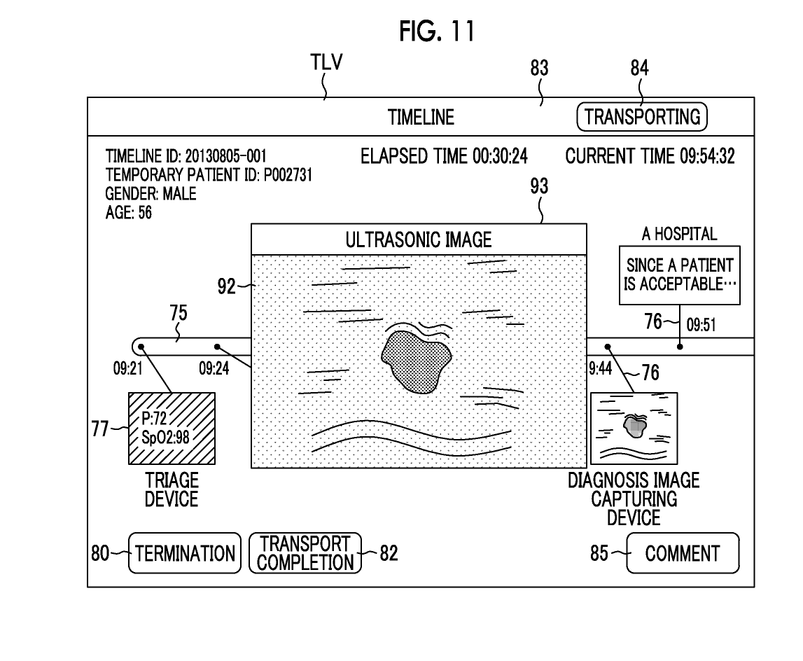

FIG. 11 illustrates a state in which the information display frame 77 of the diagnosis image capturing device has been selected in the timeline screen TLV. A sub-window 93 on which an ultrasonic image 92 is displayed is displayed on the timeline screen TLV.

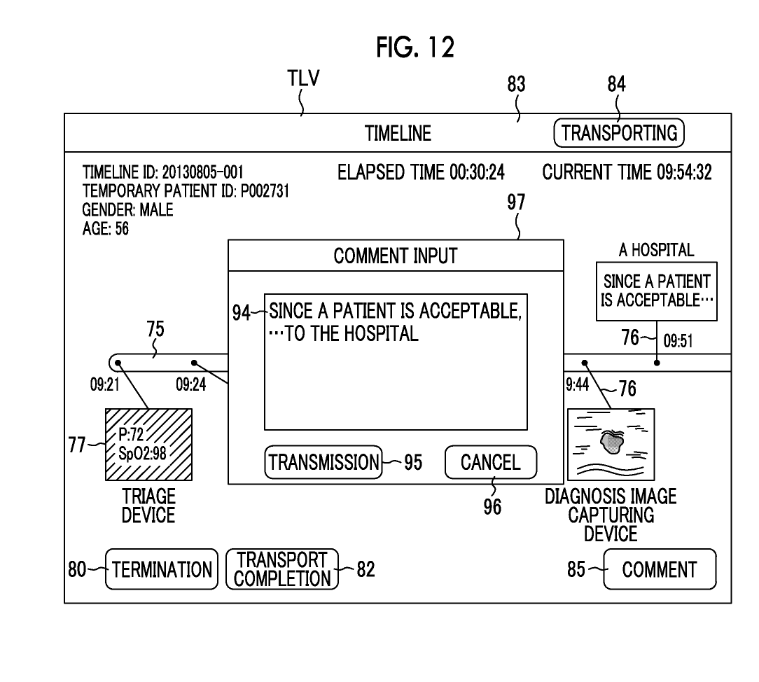

FIG. 12 illustrates a case in which the comment button 85 has been operated in the timeline screen TLV. A comment input window 97 in which a comment input field 94, a transmission button 95, and a cancel button 96 are provided is displayed on the timeline screen TLV.

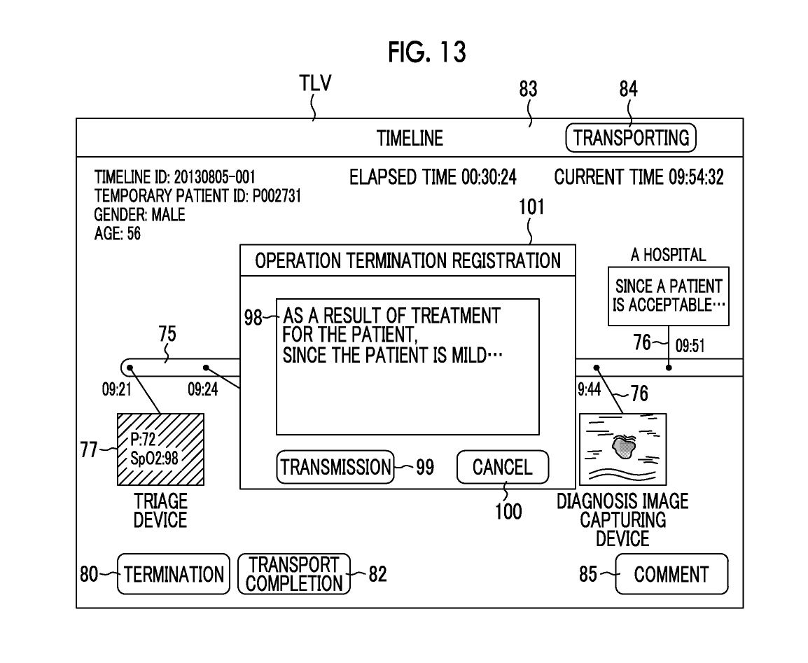

FIG. 13 illustrates a case in which the termination button 80 is operated in the timeline screen TLV. An operation termination registration window 101 in which a termination reason input field 98 in which a reason for termination of the operation of the timeline information TLD is input, a transmission button 99, and a cancel button 100 are provided is displayed on the timeline screen TLV. In the operation termination registration window 101, when the reason for termination is not input to the termination reason input field 98, the timeline termination information is not transmitted to the medical support server 21, even when the transmission button 99 is operated. Accordingly, even when the termination button 80 is erroneously operated, the timeline information TLD is not immediately terminated.

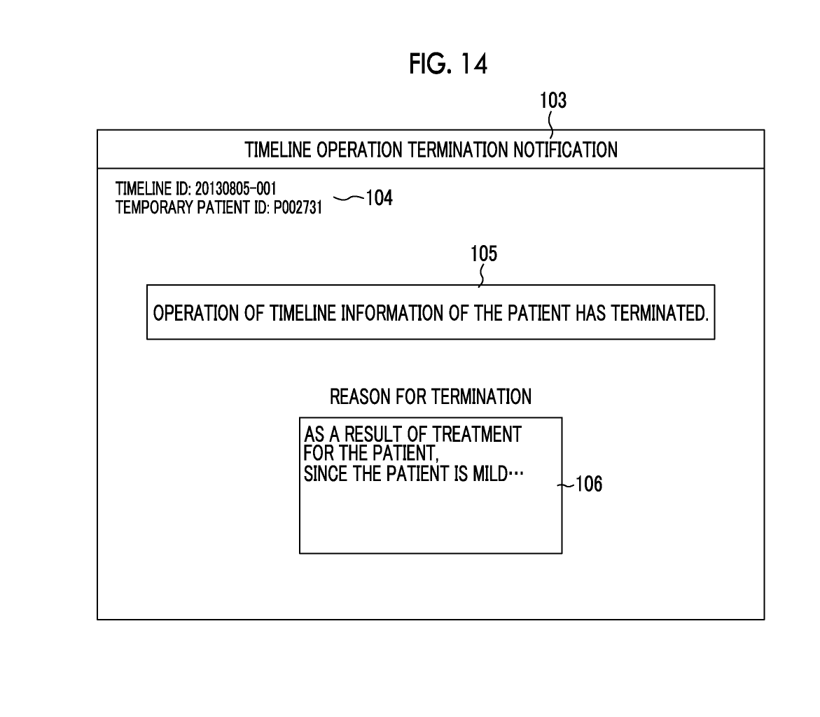

FIG. 14 illustrates an operation termination screen 103 to be distributed to the client terminals 26 to 28 after the operation termination of the timeline information TLD. A display area 104 for the timeline ID and the temporary patient ID, a display area 105 for a message indicating that the operation of the timeline information TLD terminates, and a display area 106 in which the reason for termination input in the operation termination registration window 101 is displayed, are provided in the operation termination screen 103.

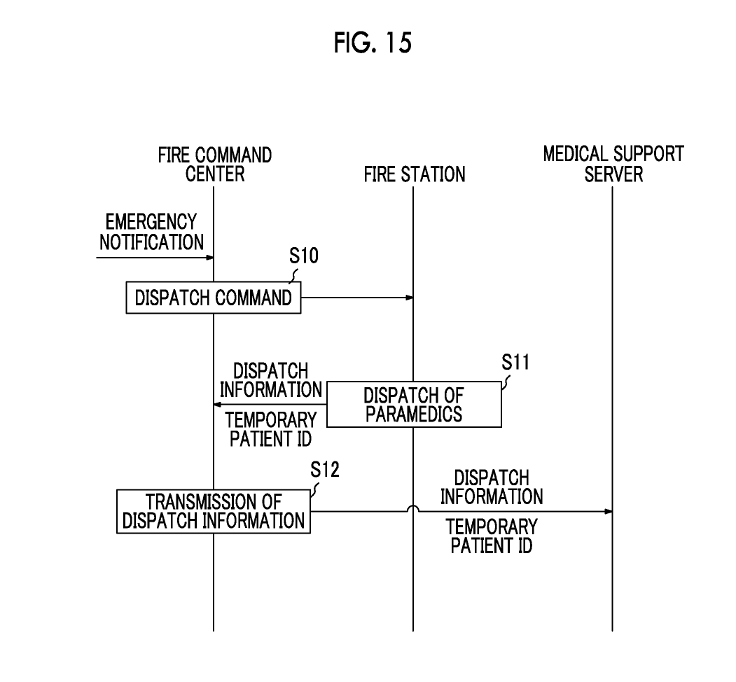

Next, an operation of the above embodiment will be described. As illustrated in FIG. 15, the fire command center 10 receives an emergency notification from the patient P or the like, and specifies an address of the site 15 from content of the notification or the like. The fire emergency system of the fire command center 10 imparts the temporary patient ID to the patient P, and transmits a dispatch command including the temporary patient ID to the fire station 11 (S10).

The fire station 11 organizes paramedics in response to the dispatch command, and dispatches the ambulance 16 (S11). The dispatch processing system of the fire station 11 transmits dispatch information including device information of each of the medical devices 22 to 25 mounted on the ambulance 16, terminal information of the emergency portable terminal 26 of each paramedic F to dispatch, and the temporary patient ID to the fire command center 10. The fire emergency system of the fire command center 10 transmits the dispatch information and the temporary patient ID to the medical support server 21 over the communication network 30 (S12).

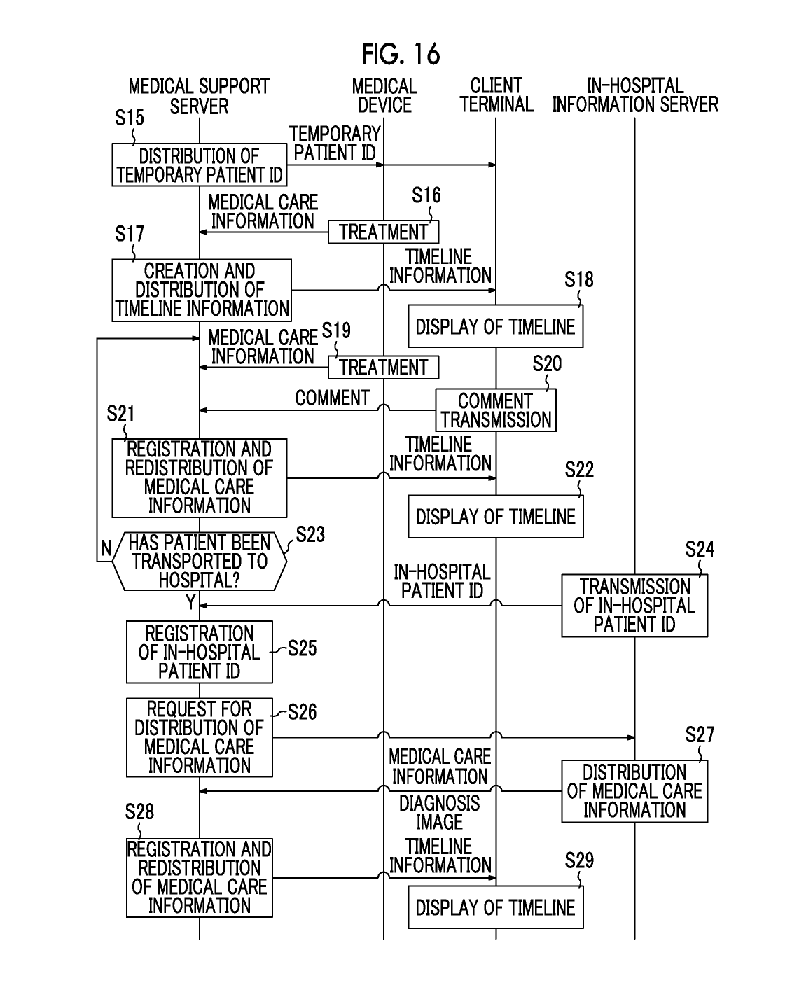

As illustrated in FIG. 16, the patient ID distribution unit 49 of the medical support server 21 distributes the temporary patient ID to each of the medical devices 22 to 25 and each emergency portable terminal 26 based on the communication information included in the dispatch information. Further, the patient ID distribution unit 49 also distributes the temporary patient ID to each in-hospital terminal 27 and each medical specialist portable terminal 28 based on the distribution destination DB 54 (S15).

Paramedics arriving at the site 15 start medical care of the patient P. The paramedic F brings out the triage device 22 from the ambulance 16, mounts the triage device 22 on a body of the patient P, and begins to set the degree of severity and measures the vital signs (S16). The triage device 22 transmits triage information Td including the degree of severity, the result of vital sign measurement, and the temporary patient ID as medical care information from the communication unit 60 to the medical support server 21 (S16).

The treatment start information reception unit 50a receives first medical care information acquired from the patient P and, in this case, the triage information. The triage information initially received by the treatment start information reception unit 50a is handled as the treatment start information. The timeline creation unit 50b starts creation of the timeline information TLD in response to reception of the treatment start information (S17). The timeline creation unit 50b registers the received medical care information and the temporary patient ID in the format information 52 read from the storage device 44 to create the timeline information TLD, imparts a timeline ID to the timeline information TLD, and stores the resultant timeline information TLD in the timeline DB 53.

When the medical device that initially acquires the medical care information from the patient P is the vital sign measurement device 23, the timeline information TLD is created according to the vital information of the vital sign measurement device 23. Further, when the medical device that initially acquires the medical care information from the patient P is the diagnosis image capturing device 24, the timeline information TLD is created according to the ultrasonic image information of the diagnosis image capturing device 24. Similarly, when the medical device that initially acquires the medical care information from the patient P is the in-vehicle camera 25, the timeline information TLD is created according to the in-vehicle imaging data of the in-vehicle camera 25.

The distribution unit 51 reads the timeline information TLD stored in the timeline DB 53 and creates the timeline screen TLV based on the read timeline information TLD. The distribution unit 51 distributes the created timeline screen TLV to the client terminals 26 to 28 based on the distribution information stored in the distribution destination DB 54 (S17). The emergency portable terminal 26, the in-hospital terminal 27, and the medical specialist portable terminal 28 receive the timeline screen TLV distributed by the distribution unit 51 and display the timeline screen TLV on the display (S18).

The paramedic F, the doctor D, and the medical specialist S can view the timeline screen TLV displayed on the display. Further, when confirmation of details of the medical care information on the timeline screen TLV is desired, the information display frame 77 is selected. Then, as illustrated FIGS. 10 and 11, a sub-window in which the selected medical care information is displayed is displayed on the timeline screen TLV.

The paramedic F continues to perform the treatment of the patient P even after the first treatment that is a creation trigger of the timeline information TLD is performed (S19). The paramedic F photographs the site 15, a state of the patient P at the site 15, or the like using the emergency portable terminal 26. Further, the paramedic F records an interview of the patient P or a witness, using the emergency portable terminal 26. The paramedic F transports the patient P into the ambulance 16 using a stretcher or the like, and then, continues to perform the treatment of the patient P using the vital sign measurement device 23 or the diagnosis image capturing device 24. Further, the in-vehicle camera 25 photographs the patient P in the ambulance 16.

The triage device 22, the vital sign measurement device 23, the diagnosis image capturing device 24, the in-vehicle camera 25, and the emergency portable terminal 26 used for treatment of the patient P transmit the triage information, the vital information, the ultrasonic image data, the in-vehicle imaging data, and portable imaging data to which the temporary patient ID has been assigned, as the medical care information, to the medical support server 21 (S19).

When the paramedic F has a question or the like for the treatment of the patient P, the paramedic F calls for the comment input window 97 on the timeline screen TLV of the emergency portable terminal 26 as illustrated in FIG. 12, and inputs the question to the comment input field 94. Further, when the doctor D and the medical specialist S perform an answering a question of the paramedic F, providing advice or an opinion, asserting acceptance of the patient P, or the like, the doctor D and the medical specialist S input such comments to the comment input window 97. The comment input to the comment input window 97 is transmitted to the medical support server 21 as the medical care information (S20). Accordingly, the paramedic F can obtain the answer to the question from the timeline information TLD, information on whether or not the patient is acceptable, or the like.

When the medical care information registration unit 50c receives the medical care information from each of the medical devices 22 to 25 and each of the client terminals 26 to 28 after the timeline information TLD is created, the medical care information registration unit 50c confirms the temporary patient ID in the received medical care information, and registers the received medical care information in the timeline information TLD corresponding to the temporary patient ID (S21). The distribution unit 51 redistributes the timeline screen TLV of the timeline information TLD in which the medical care information has been registered, to each of the client terminals 26 to 28 (S21). The emergency portable terminal 26, the in-hospital terminal 27, and the medical specialist portable terminal 28 display the redistributed timeline screen TLV on the display (S22).

When the patient P is transported to any one of the hospitals 12A to 12C, the transport completion button 82 in the timeline screen TLV is operated by the paramedic F. When the transport completion button 82 is operated, the emergency portable terminal 26 transmits transport completion information to the medical support server 21. The medical support server 21 specifies the completion of the transport of the patient P to the hospital by receiving the transport completion information. The medical care information registration unit 50c changes the display of the transport situation display portion 84 of the timeline screen TIN from “transporting” to “transported”.

The in-hospital information server 29 imparts the in-hospital patient ID to the transported patient P, and transmits this in-hospital patient ID to the medical support server 21 (S24). The medical care information registration unit 50c registers the in-hospital patient ID in the timeline information TLD of the patient P (S25).

When the patient P is transported to the hospital (YES in S23), the medical care information registration unit 50c specifies the in-hospital patient ID of the patient P and requests the in-hospital information server 29 of the hospital to which the patient P has been transported, to distribute the medical care information and the diagnosis image (S26). In response to the distribution request, the in-hospital information server 29 reads the medical care information of the patient P from the electronic medical chart DB, reads the diagnosis image of the patient P from the diagnosis image DB, and distributes the medical care information and the diagnosis image to the medical support server as medical care information (S27). The medical care information registration unit 50c registers the medical care information and the diagnosis image distributed from the in-hospital information server 29 in the timeline information TLD of the patient P (S28). The distribution unit 51 redistributes the timeline screen TLV to the in-hospital terminal 27 and the medical specialist portable terminal 28 based on the timeline information TLD in which the medical care information has been registered (S28). The in-hospital terminal 27 and the medical specialist portable terminal 28 display the redistributed timeline screen TLV on the display (S29).

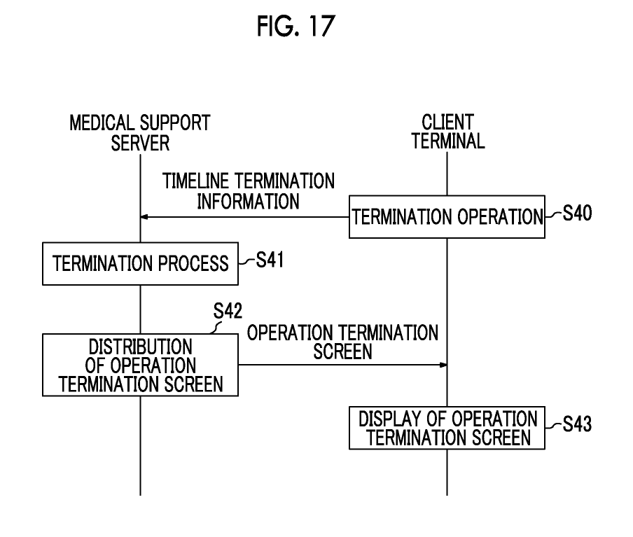

When the timeline information TLD is not required due to a condition of the patient being mild or when the timeline information TLD is not required due to patient's recovery, death, or the like, the termination button 80 of the timeline screen TLV is operated in the medical support server 21 or the client terminals 26 to 28. The operation termination registration window 101 illustrated in FIG. 13 is displayed on the display of the terminal in which the termination button 80 has been operated. As illustrated in FIG. 17, when the reason for termination is input in the operation termination registration window 101 and the transmission button 99 is operated, the timeline termination information is transmitted to the medical support server 21 (S40).

The timeline termination unit 50d receiving the timeline termination information starts the termination process (S41). In this termination process, “operation termination” and a reason for operation termination are registered in the status of the timeline information TLD of the patient that is a terminating target. For the timeline information TLD in which the status is “operation termination”, the registration of the medical care information then terminates. When the operation of the timeline information TLD terminates in the timeline termination unit 50d, the distribution unit 51 distributes the operation termination screen 103 illustrated in FIG. 14 to the client terminals 26 and 27 (S42). The operation termination screen 103 is displayed on the display of the client terminals 26 to 28 (S43). Accordingly, the paramedic F, the doctor D, and the medical specialist S can recognize that the operation of the timeline information TLD of the patient has terminated.

As described above, according to the medical support system 19 of this embodiment, since the timeline information TLD can be created in the initial emergency response step, the medical care information in the initial emergency response step can be registered in the timeline information TLD. Accordingly, it is possible to view the medical care information in the initial emergency response step from the timeline information TLD, and accordingly, to appropriately diagnose the patient by referring to the medical care information of the initial emergency response step. Further, since the timeline information TLD is created according to the treatment start information indicating that treatment for a patient who uses the medical device is started, there is no need for the duties of the paramedic to be interrupted for creation of the timeline information TLD. Furthermore, since the first medical care information acquired from the patient is used as the treatment start information, it is possible to create the timeline information TLD early in the initial emergency response step.

Further, since the temporary patient ID is imparted to the treatment start information and the timeline information TLD is created for each temporary patient ID, a plurality of pieces of timeline information TLD can be created for each patient even when there are a plurality of patients. Further, since the same number of timeline information TLD as the number of patients is created, it is possible to prevent mismanagement of the medical care information, such as medical care information of one patient being managed in a plurality of timelines data TLD, and medical care information of a plurality of patients being managed in one piece of timelines data TLD. Furthermore, since the temporary patient ID is distributed from the medical support server 21 to each of the medical devices 22 to 25 and each of the client terminals 26 to 28, the temporary patient ID can be set in each medical device and each client terminal even when a large number of medical devices 22 to 25 and a large number of client terminals 26 to 28 are used.

Further, while in this embodiment, the timeline information TLD of the patient is necessarily created when the paramedic F is dispatched, the operation of the timeline information TLD can terminate when the timeline information TLD is not required due to a condition of the patient being mild or when the timeline information TLD is no longer required due to patient's recovery, death, or the like. Accordingly, it is possible to prevent confusion or wrong medical care from being caused by continuous operation of unnecessary timeline information TLD, and to reduce a load on the medical support server 21.

In the above embodiment, the medical care information first acquired from the patient P in the medical devices such as the triage device 22, the vital sign measurement device 23, the diagnosis image capturing device 24, and the in-vehicle camera 25 has been identified as treatment start information, and the timeline information TLD has been created. On the other hand, when a type of medical device initially used in the initial emergency response step is determined in advance, the timeline information TLD may be created according to medical care information that such a type of medical device has first acquired from the patient P. In a general site of emergency, the triage device 22 brought out and used in the site is highly likely to be initially used, and accordingly, the timeline information TLD may be created based on the triage information of the triage device 22.

Further, in the above embodiment, although the terminated timeline information TLD is left in the timeline DB 53, the terminated timeline information TLD may be deleted.

In the above embodiment, since the temporary patient ID is distributed from the medical support server 21 to each of the medical devices 22 to 25 and each of the client terminals 26 to 28 when the paramedics are dispatched, only one type of temporary patient ID can be set. However, although the number of patients is one at the time of the dispatch, it may be found that there are a plurality of patients, after the paramedics arrive at the site. In this case, since one type of temporary patient ID is imparted to the plurality of patients and the same timeline information TLD is used, this may cause medical error.

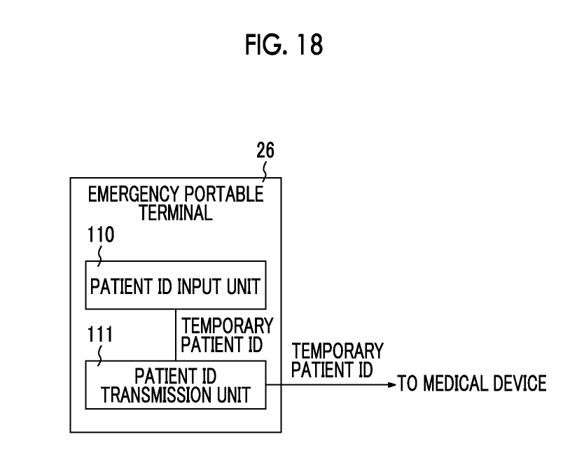

In order to be able to cope with such a case, a patient ID input unit 110 that manually inputs the temporary patient ID using a touch panel or the like, and a patient ID transmission unit 111 that individually transmits the input temporary patient ID to each medical device may be provided in the emergency portable terminal 26, as illustrated in FIG. 18. Accordingly, even when it is found that there are the plurality of patients at the site, it is possible to impart a temporary patient ID to each patient, and thus, to create the timeline information TLD for each patient.

### Second Embodiment

Next, a second embodiment of the present invention will be described. This embodiment relates to a medical support system that transfers all or part of the function of managing the timeline information TLD in the medical support server to another server after the initial emergency response step has ended, that is, after the patient P is transported to a hospital. The same configurations as those in the first embodiment are denoted with the same reference numerals, and detailed description is omitted.

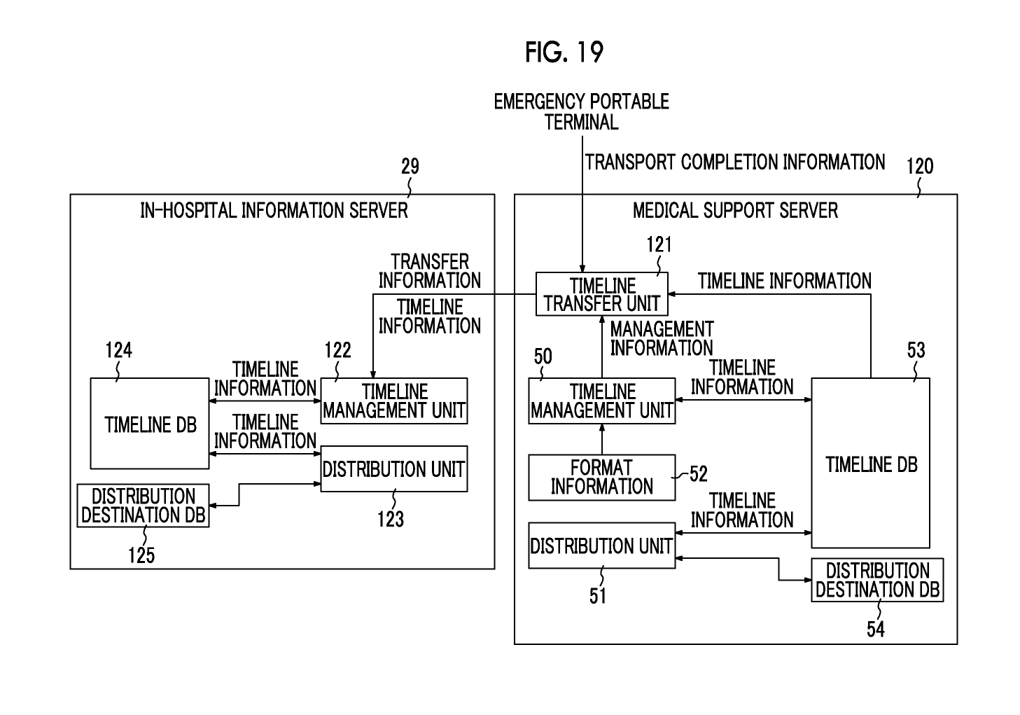

As illustrated in FIG. 19, a medical support server 120 of this embodiment includes a timeline transfer unit 121 that transfers all or part of the function of managing the timeline information TLD to another server. A server to which the function of managing the timeline information TLD is transferred is, for example, the in-hospital information server 29 of the hospital to which the patient P is transported. The in-hospital information server 29 includes a timeline management unit 122 having the same function as that of the medical support server 120, a distribution unit 123, a timeline DB 124, and a distribution destination DB 125.

When the transport completion button 82 is operated in the timeline screen TLV of the emergency portable terminal 26, the transport completion information is input from the emergency portable terminal 26 to the timeline transfer unit 121. The transport completion information is information indicating completion of the transport of the patient P to the hospital, as described in the first embodiment.

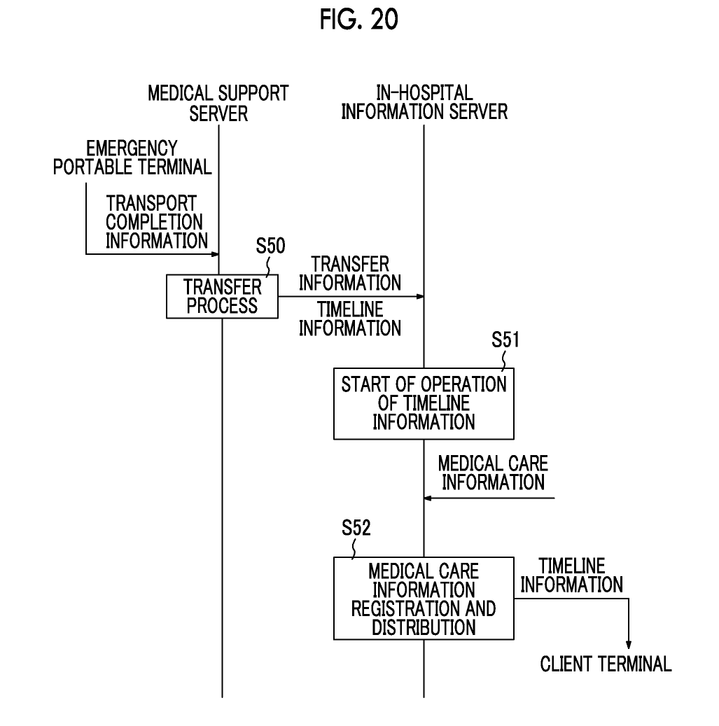

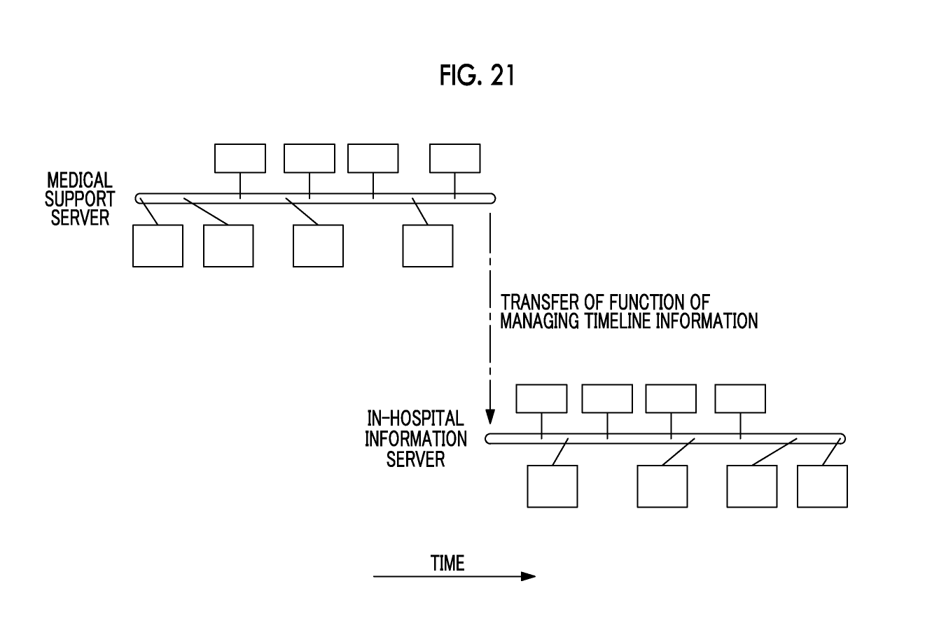

As illustrated in FIG. 20, when the transport completion information is input from the emergency portable terminal 26, the timeline transfer unit 121 executes a transfer process (S50). In this transfer process, the timeline transfer unit 121 transmits the timeline information TLD of the patient P and transfer information to the timeline management unit 122 of the in-hospital information server 29. The transfer information is information indicating that the timeline information TLD of the patient P is transferred. The timeline management unit 122 of the in-hospital information server 29 confirms that the timeline information TLD is transferred based on the transfer information, stores the received timeline information TLD in the timeline DB 124, and starts the operation of the timeline information TLD (S51). The timeline management unit 122 takes over the operation of the timeline information TLD of the patient P operated by the medical support server 120, and starts the registration of the medical care information in the timeline information TLD and the distribution of the timeline information TLD to the client terminal (S52), as illustrated in FIG. 21.

According to this embodiment, since authorization to manage the timeline information TLD of the medical support server is transferred to another server after the initial emergency response step has ended, it is possible to reduce a load on the medical support server that is used in a wide medical service zone of each administrative district.

In the second embodiment, although functions of the storage of the timeline information TLD, the registration of the medical care information, and the distribution of the timeline information TLD have been transferred to the in-hospital information server 29, at least one of these may be transferred.

While, in the above embodiment, the medical support server has been installed in the fire command center, the medical support server may be installed in an emergency medical center or a data center within the medical service zone, a data center outside the medical service zone, or the like. Further, although authorization to manage the timeline information TLD is transferred after the transport of the patient to the hospital is completed, the authorization may be transferred after a transport destination hospital is determined.

While a triage device that sets the degree of severity through a touch panel has been used as the triage device, for example, a triage tag in which a circuit in which a resistance or the like is changed according to a position at which the tag is broken, such that the degree of severity can be set, is provided in the tag having the same appearance as that of a conventional triage tag made of paper may be used.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A medical support system comprising:
a medical device used for treatment of patients; and
a medical support server that is communicably connected to the medical device over a communication network, wherein
the medical device is used by a paramedic to treat the patient in an initial emergency response step in which the patient is transported from a site as a transport source to a medical facility as a transport destination, and the medical device includes a treatment start information transmission unit for transmitting treatment start information indicating that treatment for the patient starts to the medical support server upon the medical device being used, and
the medical support server includes a treatment start information reception unit for receiving the treatment start information, and a timeline creation unit for creating timeline information for managing medical care information of the patient along a time axis in the case where the treatment start information reception unit receives the treatment start information.

2. The medical support system according to claim 1, wherein
a patient ID for identifying the patient is imparted to the treatment start information, and
the timeline creation unit creates one piece of timeline information for each patient ID.

3. The medical support system according to claim 2, wherein the patient ID is a temporary ID that is temporarily issued in the initial emergency response step.

4. The medical support system according to claim 2, wherein
the medical device includes a patient ID reception unit for receiving the patient ID input manually or the patient ID transmitted from the medical support server, and
the patient ID received by the patient ID reception unit is imparted to the treatment start information.

5. The medical support system according to claim 2, wherein
the treatment start information reception unit is adapted to receive the treatment start information from a plurality of the medical devices, and
the timeline creation unit creates the timeline information based on the initially received treatment start information among a plurality of pieces of the treatment start information including the same patient ID received by the treatment start information reception unit.

6. The medical support system according to claim 1, wherein the treatment start information reception unit receives first medical care information acquired from the patient by the medical device, as the treatment start information.

7. The medical support system according to claim 1, wherein the medical support server includes a medical care information registration unit for receiving the medical care information transmitted from the medical device and registering the received medical care information in the timeline information.

8. The medical support system according to claim 1, wherein at least one of a triage device for classifying the patient according to a degree of severity, a vital sign measurement device for measuring vital signs of the patient, and a photographing device for capturing a diagnosis image of the patient is included in the medical device.

9. The medical support system according to claim 1, wherein the medical device is adapted to be mounted on an ambulance that transports the patient to the medical facility.

10. The medical support system according to claim 7, wherein the medical support server includes a distribution unit for distributing the timeline information to a client terminal over the communication network.

11. The medical support system according to claim 10, wherein the medical care information registration unit is adapted to register information transmitted from the client terminal as the medical care information in the timeline information.

12. The medical support system according to claim 11, wherein the client terminal is used by the paramedic or doctor to input a comment to the timeline information.

13. The medical support system according to claim 1, wherein the medical support server includes a timeline termination unit for performing a termination process of terminating an operation of the timeline information.

14. The medical support system according to claim 7, wherein the medical support server includes a timeline transfer unit for transferring all or part of a management function of managing the timeline information to another server after the initial emergency response step has ended.

15. The medical support system according to claim 14, wherein the timeline transfer unit transfers at least one of a storage destination for the timeline information, and a registration right for the medical care information.

16. The medical support system according to claim 14, wherein the other server as a transport destination for the timeline information is an in-hospital server managed by the medical facility as the transport destination of the patient.
